(19) United States
(12) Patent Application Publication
(10) Pub. No.: US 2018/0210632 A1
(43) Pub. Date: Jul. 26, 2018

(54) METHOD AND ULTRASOUND IMAGING SYSTEM FOR ADJUSTING AN ULTRASOUND IMAGE WITH A TOUCH SCREEN

(71) Applicant: General Electric Company, Schenectady, NY (US)

(72) Inventors: Heinz Schmied, Mondsee (AT); Balint Czupi, Seewalchen am Attersee (AT)

(21) Appl. No.: 15/410,981

(22) Filed: Jan. 20, 2017

(51) Int. Cl. G06F 3/0484; A61B 8/00; G06F 3/0481; G06F 3/0488; G06T 19/20

(52) U.S. Cl. CPC G06F 3/04845; G06F 2203/04803; A61B 8/467; A61B 8/54; A61B 8/4444; G06F 3/04817; G06F 3/04883; G06T 19/20; G06F 3/04842; G06F 3/04815; G06F 3/04886; G06F 2203/04808; G06T 2219/2016; G06F 2203/04806; A61B 8/461

(57) ABSTRACT

An ultrasound imaging system and a method for controlling a diagnostic imaging system are disclosed. The system includes a probe, a main screen, a touch screen and a processor connected to the probe, the main screen and the touch screen. The processor is configured to display an ultrasound image on the main screen, display graphical user interface icons on the touch screen, and to perform a command associated with one of the graphical user interface icons on the touch screen while in a standard mode. The processor is configured to switch from operating in the standard mode to operating in an image-manipulation mode in response to receiving a multi-touch gesture through the touch screen. In the image-manipulation mode, the multi-touch gesture is used to adjust the ultrasound image.

### FIELD OF THE INVENTION

This disclosure relates to an ultrasound imaging system with both a main screen and a touch screen and a method for switching between a standard mode and an image-manipulation mode to adjust an ultrasound image displayed on the main screen with the touch screen.

### BACKGROUND OF THE INVENTION

Some conventional ultrasound imaging systems have two separate screens: a main screen configured for displaying ultrasound images and a separate touch screen that is responsive to touch-based inputs. The touch-based inputs may be either single-touch inputs or multi-touch inputs.

In conventional ultrasound imaging systems, the touch screen is typically used to interacting with a plurality of graphical user interface icons displayed on the touch screen. The user may use single-touch or multi-touch gestures to initiate one or more actions by selecting various graphical user interface icons.

It would also be desirable to use the touch screen to directly manipulate ultrasound images displayed on the main screen. For example, the user may wish to perform operations such as rotating an image, changing a scale of an image, or translating an image. However, in conventional systems, the touch screen is typically configured only for interacting with the graphical user interface icons. Therefore, there exists a need for an improved diagnostic imaging system and method for adjusting ultrasound images on ultrasound imaging systems with both a main screen and a touch screen. Specifically, there is a need for an easy and intuitive way to use a touch screen to both directly adjust an ultrasound image displayed on the main screen and to interact with graphical user interface icons displayed on the same touch screen.

### BRIEF DESCRIPTION OF THE INVENTION

The above-mentioned shortcomings, disadvantages, and problems are addressed herein, which will be understood by reading and understanding the following specification.

In an embodiment, a method of controlling an ultrasound imaging system including both a main screen and a separate touch screen includes displaying an ultrasound image on the main screen and displaying graphical user interface icons on the touch screen at the same time as the ultrasound image is displayed on the main screen while the ultrasound imaging system is in a standard mode. Where in the standard mode, the touch screen is used to interface with the graphical user interface icons on the touch screen. Receiving a single-touch gesture through the touch screen while in the standard mode and performing a command associated with one of the graphical user interface icons in response to the single-touch gesture while in the standard mode. Receiving a multi-touch gesture through the touch screen and switching from operating in the standard mode to operating in an image-manipulation mode in response to the multi-touch gesture, wherein, in the image-manipulation mode, the touch screen is used to directly adjust the ultrasound image displayed on the main screen, and directly adjusting the ultrasound image based on the multi-touch gesture while in the image-manipulation mode.

In an embodiment, an ultrasound imaging system includes a probe configured to acquire ultrasound data, a main screen, a touch screen and a processor connected to the probe, the main screen and the touch screen. The processor is configured to display an ultrasound image on the main screen, where the ultrasound image is based on the ultrasound data. The processor is configured to display graphical user interface icons on the touch screen at the same time as the ultrasound image is displayed on the main screen while operating in a standard mode, where, in the standard mode, the touch screen is used to interface with the graphical user icons on the touch screen. The processor is configured to perform a command associated with one of the graphical user interface icons in response to receiving a single-touch gesture through the touch screen while operating in the standard mode. The processor is configured to switch from operating in the standard mode to operating in an image-manipulation mode in response to receiving a multi-touch gesture through the touch screen. Where, in the image-manipulation mode, the touch screen is used to directly adjust the ultrasound image displayed on the main screen. The processor is configured to directly adjust the ultrasound image displayed on the main screen in response to the multi-touch gesture.

In an embodiment, a non-transitory computer readable medium having stored thereon, a computer program having at least one code section, the at least one code section being executable by a machine for causing the machine to perform steps including displaying an ultrasound image on a main screen of an ultrasound imaging device while displaying graphical user interface icons on a touch screen of the diagnostic imaging device in a standard mode. Where, in the standard mode, the touch screen is used to interface with the graphical user interface icons. Performing a command associated with one of the graphical user interface icons in response to receiving a single-touch gesture selecting one of the GUI icons while in the standard mode. Switching from operating in the standard mode to operating in an image-manipulation mode in response to receiving a multi-touch gesture through the touch screen. Where, in the image-manipulation mode, the touch screen is used to directly adjust the ultrasound image displayed on the main screen. Directly adjusting the ultrasound image based on the multi-touch gesture.

Various other features, objects, and advantages of the invention will be made apparent to those skilled in the art from the accompanying drawings and detailed description thereof.

### BRIEF DESCRIPTION OF THE DRAWINGS

FIG. 1 is a schematic representation of an ultrasound imaging system in accordance with an embodiment;

FIG. 2 is a schematic representation of a perspective view of an ultrasound imaging system in accordance with an embodiment;

FIG. 3 is a flow chart showing a method in accordance with an embodiment;

FIG. 4 is a schematic representation of a touch screen including a plurality of graphical user interface icons in accordance with an embodiment;

FIG. 5 is schematic representation of a touch screen and a main screen according to an embodiment where a multi-touch input comprises spreading two fingers apart from each other;

FIG. 6 is a schematic representation of a touch screen and a main screen according to an embodiment where a multi-touch input comprises rotating two fingers;

FIG. 7 is schematic representation of a touch screen and a main screen in an embodiment where a multi-touch input comprises translating two fingers; and

FIG. 8 is a schematic representation of a touch screen and a main screen according to an embodiment where a two icons are positioned on the main screen to correspond to the multi-touch gesture performed on the touch screen.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical, and other changes may be made without departing from the scope of the embodiments. The following detailed description is, therefore, not to be taken as limiting the scope of the invention.

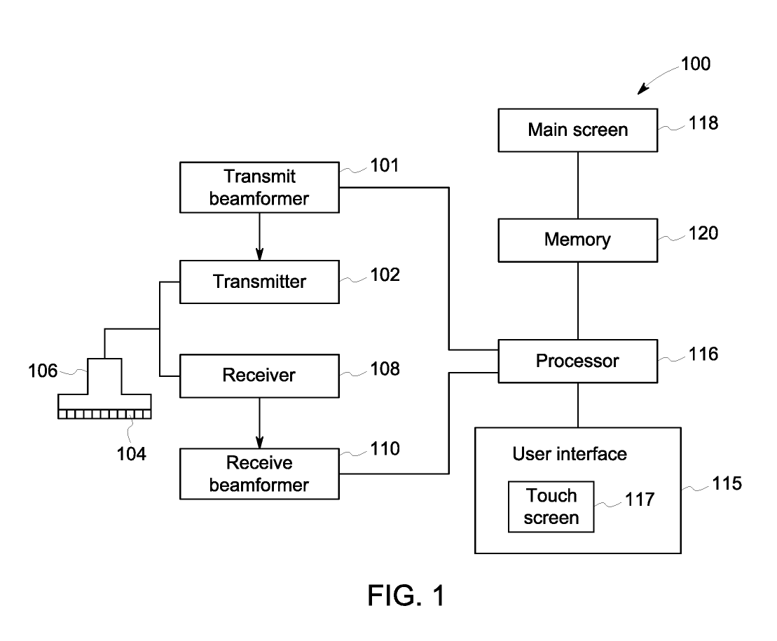

FIG. 1 is schematic diagram of ultrasound imaging system 100. The ultrasound imaging system 100 includes a transmit beamformer 101 and a transmitter 102 that drive elements 104 within a probe 106 to emit pulsed ultrasonic signals into a body (not shown). According to an embodiment, the probe 106 may be capable of acquiring real-time 3D ultrasound images. For example, the probe 106 may be a mechanical probe that sweeps or oscillates an array in order to acquire the real-time 3D ultrasound data, or the probe 106 may be a 2D matrix array with full beam-steering in both the azimuth and elevation directions. Still referring to FIG. 1, the pulsed ultrasonic signals are back-scattered from structures in the body, like blood cells or muscular tissue, to produce echoes that return to the elements 104. The echoes are converted into electrical signals, or ultrasound data, by the elements 104, and the electrical signals are received by a receiver 108. The electrical signals representing the received echoes are passed through a receive beamformer 110 that outputs ultrasound data. According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The terms “data” and “ultrasound data” may be used in this disclosure to refer to either one or more datasets acquired with an ultrasound imaging system. A user interface 115 may be used to control operation of the ultrasound imaging system 100. The user interface 115 may be used to control the input of patient data, or to select various modes, operations, and parameters, and the like. The user interface 115 includes a touch screen 117 and may additionally include a one or more user input devices such as a keyboard, hard keys, a track ball, rotary controls, sliders, soft keys, or any other user input devices. The ultrasound imaging system 100 also includes a main screen 118 for displaying ultrasound images. The touch screen 117 is a multi-touch screen that can detect either single-touch gestures or multi-touch gestures. Multi-touch gestures are gestures input through the touch screen 117 that involve two or more distinct points of contact with the touch screen at the same time. Multi-touch gestures may be input with two or more fingers, for example. The touch screen 117 is a separate component from the main screen 118. However, according to some embodiments, the touch screen 117 may also be configured to display an image. The touch screen 117 may comprise light emitting diodes (LEDs), organic light emitting diodes (OLEDs), or other types of elements capable of being controlled for the purpose of forming an image. The touch screen 117 may also include one or more touch-sensitive elements such as a capacitive sensor, a resistive sensor, a pressure sensor, or any other types of sensors configured to detect when an object, such as a user's finger, is in contact with the touch screen 117.

The ultrasound imaging system 100 also includes a processor 116 to control the transmit beamformer 101, the transmitter 102, the receiver 108, and the receive beamformer 110. The receive beamformer 110 may be either a conventional hardware beamformer or a software beamformer according to various embodiments. If the receive beamformer 110 is a software beamformer, it may comprise one or more of the following components: a graphics processing unit (GPU), a microprocessor, a central processing unit (CPU), a digital signal processor (DSP), or any other type of processor capable of performing logical operations. The receive beamformer 110 may be configured to perform conventional beamforming techniques as well as techniques such as retrospective transmit beamforming (RTB).

The processor 116 is in electronic communication with the probe 106. The processor 116 may control the probe 106 to acquire ultrasound data. The processor 116 controls which of the elements 104 are active and the shape of a beam emitted from the probe 106. The processor 116 is also in electronic communication with the main screen 118, and the processor 116 may process the ultrasound data into images for display on the main screen 118. For purposes of this disclosure, the term “electronic communication” may be defined to include both wired and wireless connections. The processor 116 may include a central processing unit (CPU) according to an embodiment. According to other embodiments, the processor 116 may include other electronic components capable of carrying out processing functions, such as a digital signal processor, a field-programmable gate array (FPGA), a graphics processing unit (GPU), or any other type of processor. According to other embodiments, the processor 116 may include multiple electronic components capable of carrying out processing functions. For example, the processor 116 may include two or more electronic components selected from a list of electronic components including: a central processing unit (CPU), a digital signal processor (DSP), a field-programmable gate array (FPGA), and a graphics processing unit (GPU). According to another embodiment, the processor 116 may also include a complex demodulator (not shown) that demodulates the RF data and generates raw data. In another embodiment the demodulation may be carried out earlier in the processing chain. The processor 116 may be adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. Real-time volume rates may vary based on the size of the volume from which data is acquired and the specific parameters used during the acquisition. The data may be stored temporarily in a buffer during a scanning session and processed in less than real-time in a live or off-line operation. Some embodiments of the invention may include multiple processors (not shown) to handle the processing tasks. For example, an embodiment may use a first processor to demodulate and decimate the RF signal and a second processor to further process the data prior to displaying an image. It should be appreciated that other embodiments may use a different arrangement of processors. For embodiments where the receive beamformer 110 is a software beamformer, the processing functions attributed to the processor 116 and the software beamformer hereinabove may be performed by a single processor, such as the receive beamformer 110, or the processor 116. Or the processing functions attributed to the processor 116 and the software beamformer may be allocated in a different manner between any number of separate processing components.

According to an embodiment, the ultrasound imaging system 100 may continuously acquire real-time 3D ultrasound data at a volume-rate of, for example, 10 Hz to 30 Hz. A live ultrasound image may be generated based on the real-time 3D ultrasound data. The live ultrasound image may be refreshed at a frame-rate that is similar to the volume-rate according to an embodiment. Other embodiments may acquire data and or display the live ultrasound image at different volume-rates and/or frame-rates. For example, some embodiments may acquire real-time 3D ultrasound data at a frame-rate of less than 10 Hz or greater than 30 Hz depending on the size of the volume and the intended application. Other embodiments may use 3D ultrasound data that is not real-time 3D ultrasound data. A memory 120 is included for storing processed frames of acquired data. In an exemplary embodiment, the memory 120 is of sufficient capacity to store frames of ultrasound data acquired over a period of time at least several seconds in length. The frames of data are stored in a manner to facilitate retrieval thereof according to its order or time of acquisition. The memory 120 may comprise any known data storage medium. In embodiments where the 3D ultrasound data is not real-time 3D ultrasound data, the 3D ultrasound data may be accessed from the memory 120, or any other memory or storage device. The memory or storage device may be a component of the ultrasound imaging system 100, or the memory or storage device may external to the ultrasound imaging system 100.

Optionally, embodiments of the present invention may be implemented utilizing contrast agents and contrast imaging. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component, and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 (e.g., B-mode, Color Doppler, M-mode, Color M-mode, spectral Doppler, Elastography, TVI, strain, strain rate and combinations thereof, and the like) to form 2D or 3D images or data. For example, one or more modules may generate B-mode, color Doppler, M-mode, color M-mode, spectral Doppler, Elastography, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from beam space coordinates to display space coordinates. A video processor module may be provided that reads the image frames from a memory and displays the image frames in real time while a procedure is being carried out on a patient. A video processor module may store the image frames in an image memory, from which the images are read and displayed.

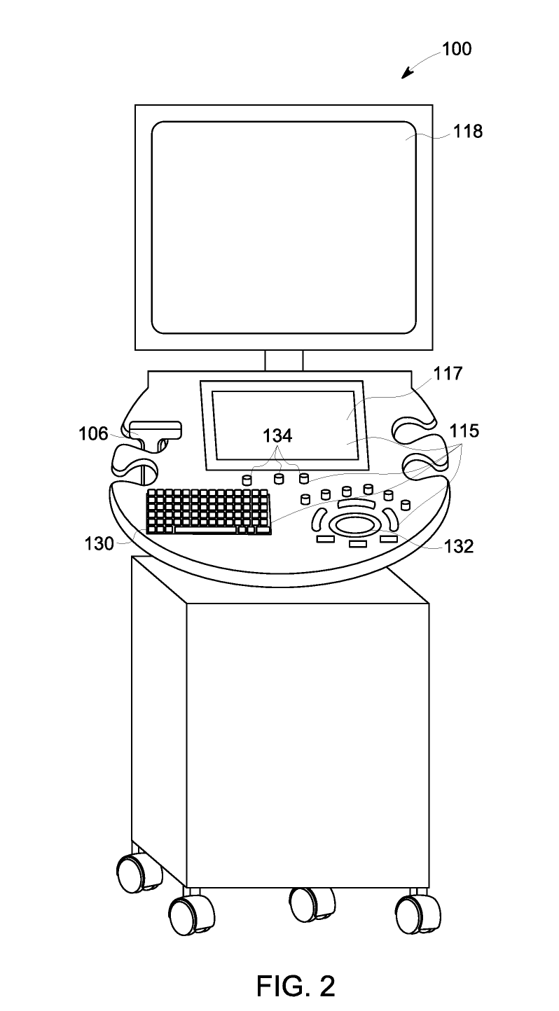

FIG. 2 is a schematic representation of a perspective view of the ultrasound imaging system 100. FIG. 2 shows an exemplary embodiment for an arrangement of the main screen 118 and the touch screen 117. The touch screen 117 is positioned within easy reach of an operator, while the main screen 118 is positioned for ergonomic viewing of ultrasound images generated from ultrasound data. In addition to the touch screen 117, the user interface 115 in the embodiment shown in FIG. 2 also includes a keyboard 130, a trackball 132 and rotary controls 134. The main screen 118 is configured to display images generated from ultrasound data. The touch screen 117 is positioned closer to the other controls of the user interface 115. The touch screen 117 may display graphical user interface icons or ultrasound images, as will be described in detail hereinafter.

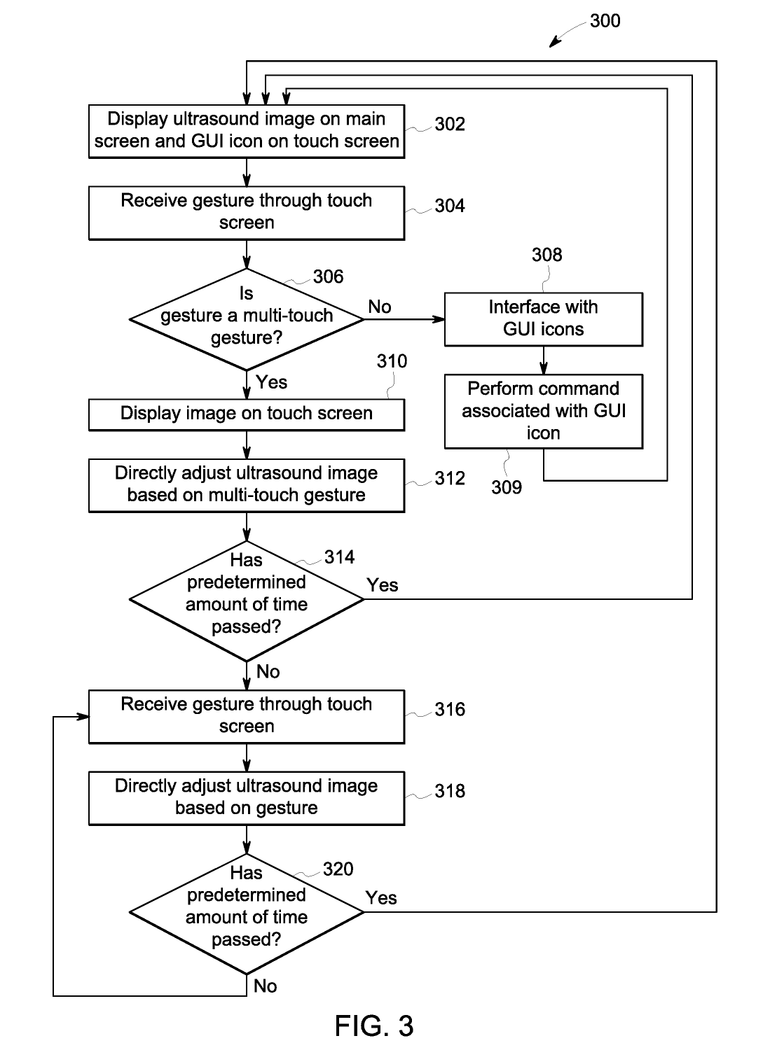

FIG. 3 is a flow chart of a method 300 in accordance with an embodiment. The method 300 will be described with respect to the ultrasound imaging system 100 shown in FIGS. 1 and 2 in accordance with an exemplary embodiment.

At step 302, the processor 116 displays an ultrasound image on the main screen 118 and a plurality of graphical user interface icons (GUI icons) on the touch screen while the ultrasound imaging system is in a standard mode. While in the standard mode, single-touch gestures input through the touch screen 117 are used to interact with the GUI icons displayed on the touch screen 117. The GUI icons may represent commands related to controlling the imaging acquisition. For example, the GUI icons may be used to initiate actions in order to control acquisition parameters such as line density, pulse repetition frequency (PRF), focal depth, or frequency, for instance. Some of the GUI icons may also represent commands related to the display of the ultrasound image on the main screen 118. For example, the GUI icons may be used to control settings such as brightness, contrast, window width, window level of the ultrasound image displayed on the main screen 118. It should be appreciated that the GUI icons may be used to control other parameters associated with the image acquisition or imaging protocol and that the GUI icons may be used to control other parameters associated with the display of the ultrasound image according to various embodiments.

There may be more GUI icons than would easily fit on the touch screen 117 at a single time. In order to manage this issue, the GUI icons displayed on the touch screen 117 may be arranged or displayed on multiple pages or screens. For example, the user may use gestures to access additional pages of GUI icons or individual GUI icons may be limited to menus used to access additional GUI icons that are commands to control acquisition or display parameters for the ultrasound imaging system 100. While in the standard mode, all gestures input through the touch screen 117 are used to interact with the GUI icons. Interacting with the GUI icons may comprise selecting/interacting with one or more GUI icons to control/adjust individual parameters. Interacting with the GUI icons may also comprise turning pages/scrolling the view on the touch screen 117 to view GUI icons that are not currently being displayed on the touch screen 117 or selecting one or more GUI icons to access additional GUI icons through a series of menus and/or sub menus.

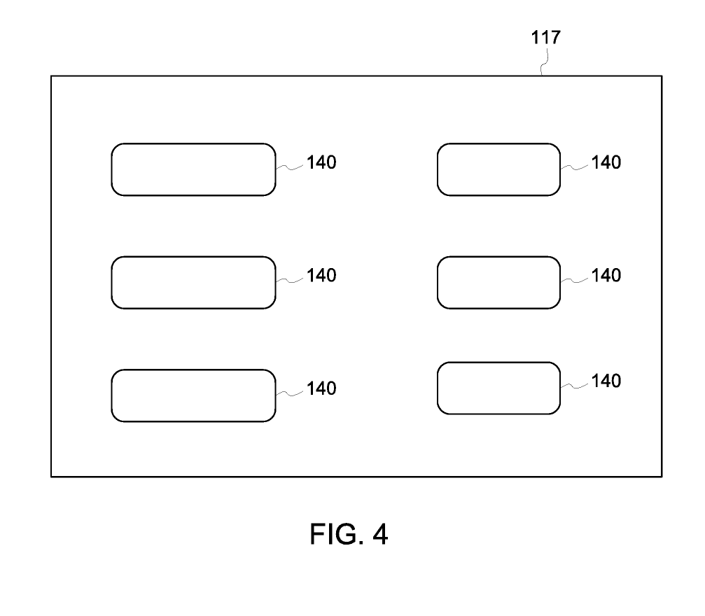

FIG. 4 is a schematic representation of the touch screen 117 with a plurality of GUI icons 140 according to an embodiment. As discussed hereinabove, each GUI icon 117 may be used to control an image acquisition parameter, an image display parameter, and/or one or more of the GUI icons may be used to access menus of additional GUI icons. According to an exemplary embodiment, the user may only interact with the GUI icons in the standard mode through single-touch gestures. Single-touch gestures are well-known by those skilled in the art and include gestures that are performed with only a single point of contact with the touch screen 117 at a time. A user might, for instance, perform a single-touch gesture using only a single finger, such as their index finger (or any other single point of contact with the touch screen 117). Examples of single touch gestures including tapping, swiping, dragging, or drawing a free-form shape on the touch screen 117.

In addition to single-touch gestures, the touch screen 117 is configured to detect multi-touch gestures. Multi-touch gestures are also well-known by those skilled in the art and are gestures performed by contacting the touch screen 117 with more than one point of contact at a time. It is possible to perform multi-touch gestures that are more complicated than single-touch gestures. A non-limiting list of multi-touch gestures includes a swipe with two or more fingers, a pinching of two or more fingers, a spreading of two or more fingers, a rotation of two or more fingers, as well as free form drawing with two or more fingers. The multi-touch gestures may be used to directly adjust the ultrasound image displayed on the main screen. For purposes of this disclosure, directly adjusting the ultrasound image includes adjusting a position, an orientation, or any other parameter related to the display of the image on the main screen 118 with the multi-touch gesture. Directly adjusting the ultrasound image additionally includes adjusting the ultrasound image without interfacing with any of the GUI icons. In other words, the inputting of the multi-touch gesture is interpreted as a command by the processor 116 to adjust the position, the orientation or another parameter related to the display of the image on the main screen 118. For example, a swipe may be used to translate an image, a pinching gesture may be used to zoom out the image, a spreading of two or more fingers may be used to zoom in the image, and a rotation of two or more fingers may be used to rotate the image. It should be appreciated that different actions resulting in directly adjusting the ultrasound image may be associated with the above list of multi-touch gestures. Additionally, other multi-touch gestures may be recognized by the processor 116 and/or the previously mentioned actions may be associated with different gestures. According to an embodiment, the user may map various actions for directly adjusting the ultrasound image to specific multi-touch gestures. For the purposes of this disclosure, the term directly adjusting includes adjusting the display of an ultrasound image in response to a multi-touch gesture without interacting with a GUI icon or other hard control. In other words, directly adjusting means that the appearance of the ultrasound image is adjusted directly in response to a multi-touch gesture input through the touch screen 117.

At step 304, the touchscreen 117 receives the gesture input from the user and the processor 116 interprets the gesture. At step 306, the processor determines if the gesture is a multi-touch gesture. If the gesture is a single-touch gesture, the process 300 advances to step 308 and the single-touch gesture is used to interface with the GUI icons. Interfacing with the GUI icons may comprise selecting a GUI icon or it may also include scrolling the view on the touch screen 117 or otherwise turning a page to view additional or different GUI icons. At step 309 the processor performs a command associated with one of the GUI icons. As was described hereinabove, the single-touch gesture may, for instance, be used to activate a command associated with a GUI icon, such as adjusting acquisition parameters, display parameters of the ultrasound image, or the single-touch gestures may be used to access a menu associated with a specific GUI icon. After step 309, the method 300 returns to step 302. According to an embodiment, steps 302, 304, 306, 308 and 309 may be iterated as many times as desired until a multi-touch gesture is received through the touch screen.

If, at step 306, the gesture is a multi-touch gesture, then the processor 116 switches the ultrasound imaging system from the standard mode to an image-manipulation mode. The processor 116 automatically enters the image-manipulation mode upon detecting a multi-touch gesture. In the image-manipulation mode, the multi-touch gesture is used to directly adjust the ultrasound image displayed on the main screen. As discussed previously, directly adjusting the ultrasound image means that the ultrasound image displayed on the main screen 118 is adjusted in terms of position, orientation, or any other display parameter in response to the detected multi-touch gesture. Upon detection a multi-touch gesture, the processor 116 switches from the standard mode, where single-touch gestures are used to interact with the GUI icons on the touch screen to the image-manipulation mode, where the multi-touch gesture is used to directly adjust the ultrasound image instead of interacting with the GUI icons on the touch screen. According to other embodiments, a specific multi-touch gesture may be used to initiate the transition from the standard mode to the image-manipulation mode. For example, some embodiments may use a multi-touch gesture such as a 3-finger drag to switch from the standard mode to the image-manipulation mode. For example, the 3-finger drag may be from the top of the touch screen 117 in a downward direction. It should be appreciated that the 3-finger drag is just one example of a multi-touch gesture that may be used to initiate the transition from the standard mode to the image-manipulation mode and that other multi-touch gestures may be used in other embodiments to initiate the transitions from the standard mode to the image-manipulation mode. Additionally, the multi-touch gesture that is used to initiate the transition from standard mode to image-manipulation mode may be user-selectable according to other embodiments.

If the gesture is a multi-touch gesture at step 306, the method 300 advances to step 310 according to an exemplary embodiment. At step 310, the processor 116 displays the same ultrasound image that is currently being displayed on the main screen 118 on the touch screen 117. The ultrasound image being displayed on the touch screen 117 may be a smaller scale representation of the ultrasound image displayed on the main screen 118, as the touch screen 117 is often smaller than the main screen 118. According to other embodiments, the ultrasound image displayed on the touch screen 117 may be the same scale as the ultrasound image displayed on the main screen 118. Other embodiments may skip step 310 and the method may advance directly from step 306 to 312 if a multi-touch gesture is detected at step 306.

At step 312, the processor directly adjusts the ultrasound image displayed on the main screen 118 based on the detected multi-touch gesture. In other words, the multi-touch gesture that causes the ultrasound imaging system 100 to transition from the standard mode to the image-manipulation mode is used to directly adjust the ultrasound image displayed on the main screen 118.

Once in the image-manipulation mode, according to some embodiments, the processor 116 may use either single-touch gestures or multi-touch gestures to directly adjust the ultrasound image. For example, after the detected multi-touch gesture causes the processor 116 to enter the image-manipulation mode, the user may use single-touch gestures and/or multi-touch gestures to directly adjust the ultrasound image displayed on the main screen 118.

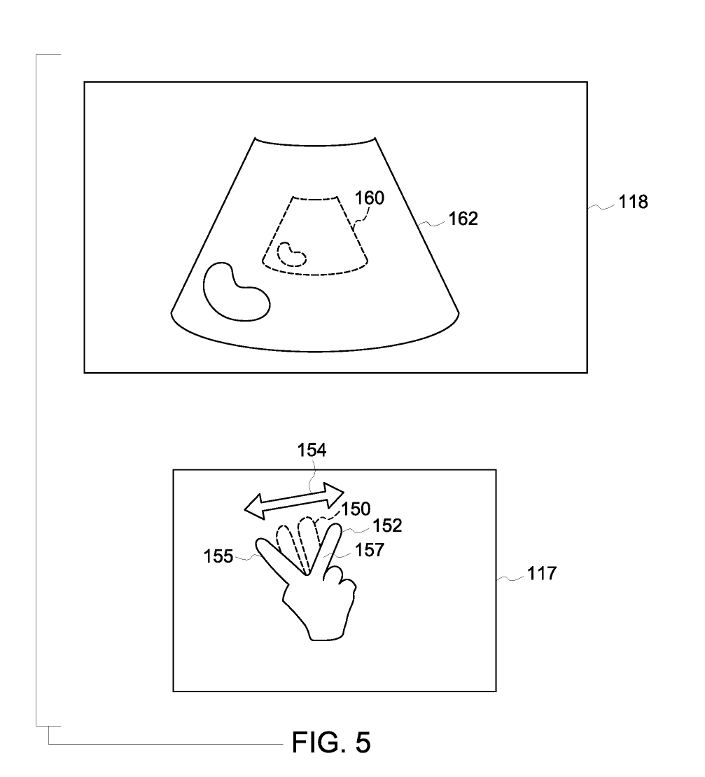

FIGS. 5, 6, and 7 are schematic representations showing exemplary multi-touch gestures that may be input through the touch screen 117 and the corresponding adjustments that may be performed on the ultrasound image displayed on the main screen 118 according to various embodiments. Certain conventions are used in each of FIGS. 5, 6, and 7: the dashed image of the hand represents a starting position of a specific multi-touch gesture and the solid line image of the hand represents an end position of the specific multi-touch gesture. Likewise, the dashed ultrasound image displayed on the main screen represents a starting position of the ultrasound image before the multi-touch gesture is performed and the solid line image of the ultrasound image represents an end position of the ultrasound image after the multi-touch gesture has been completed.

FIG. 5 is schematic representation of a touch screen and a main screen according to an embodiment where a multi-touch input comprises spreading two fingers apart from each other. FIG. 5 includes the touch screen 117 and the main screen 118. On the touch screen 117 the multi-touch gesture comprises spreading two fingers apart from each other. A starting position 150 of the hand/fingers is shown in dashed line and a final position 152 is shown in solid line. An arrow 154 is used to show the relative movement of the fingers when performing this particular multi-touch gesture. Spreading a first finger 155 apart from a second finger 157, or more than two fingers according to other embodiments, results in directly adjusting the ultrasound image by zooming in on the ultrasound image. For example an initial ultrasound image 160 is shown in dashed line and a final ultrasound image 162 is shown in solid line. The initial ultrasound image 160 represents the ultrasound image that would be displayed before the multi-touch gesture is input through the touch screen 117. The final ultrasound image 162 represents the ultrasound image that is displayed after completing the multi-touch gesture, which in this exemplary embodiment comprises spreading two fingers apart from each other. It should be appreciated that the image displayed on the main display 118 may be adjusted from the initial ultrasound image 160 to the final ultrasound 162 in real-time as the multi-touch gesture of spreading two fingers apart from each other is input through the touch screen 117. It should be appreciated by those skilled in the art that the images displayed on the main screen 118 may smoothly transition from the initial image 160 to the final image 162. In other words, intermediate images may be displayed as the multi-touch gesture is being performed on the touch screen 117 so that the ultrasound image appears to smoothly increase in size as the two fingers are spread further apart from each other on the touch screen 117. According to the embodiment represented in FIG. 5, the multi-touch gesture of spreading two fingers apart from each other results in zooming in the ultrasound image displayed on the main screen; the final image ultrasound image 162 is clearly larger (i.e., more zoomed-in) than the initial ultrasound image 160.

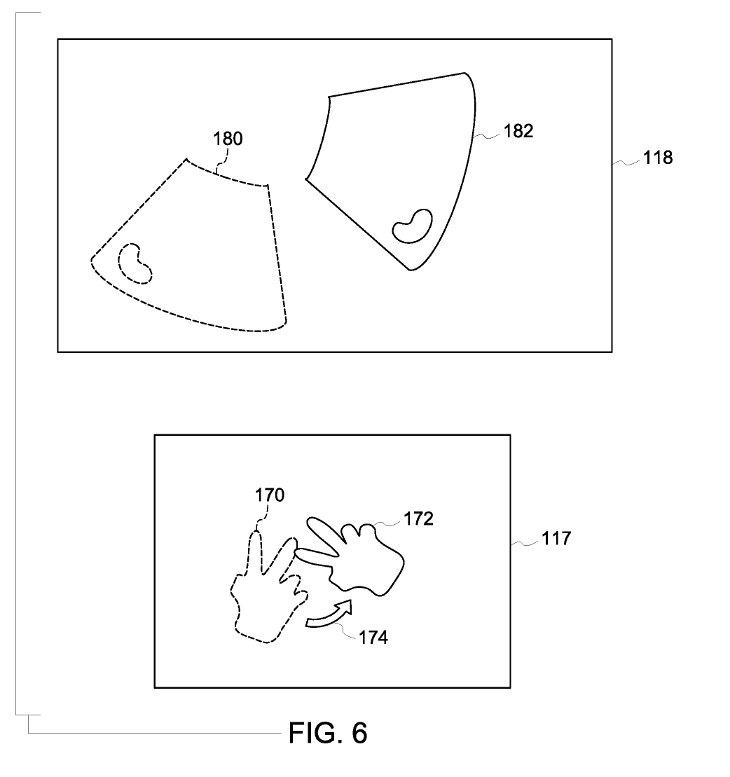

FIG. 6 is a schematic representation of a touch screen and a main screen in an embodiment where a multi-touch input comprises rotating two fingers. FIG. 6 includes the touch screen 117 and the main screen 118. On the touch screen 117 the multi-touch gesture comprises rotating two fingers. A starting position 170 of the hand/fingers is shown in dashed line and a final position 172 of the hand/fingers is shown in solid line. An arrow 174 is used to show the relative movement of the fingers when performing this particular multi-touch gesture. Rotating two fingers, or more than two fingers according to other embodiments, results in directly adjusting the ultrasound image by rotating the ultrasound image in a corresponding direction. For example an initial ultrasound image 180 is shown in dashed line and a final ultrasound image 182 is shown in solid line. The initial ultrasound image 180 represents the ultrasound image that would be displayed before the multi-touch gesture is input through the touch screen 117. The final ultrasound image 182 represents the ultrasound image that is displayed after completing the multi-touch gesture, which in this exemplary embodiment is rotating two fingers. It should be appreciated that image displayed on the main display 118 may be adjusted from the initial ultrasound image 180 to the final ultrasound 182 in real-time as the multi-touch gesture of rotating the two fingers is input through the touch screen 117. It should be appreciated by those skilled in the art that the images displayed on the main screen 118 may smoothly transition from the initial image 180 to the final image 182. In other words, intermediate images may be displayed as the multi-touch gesture is being inputted through the touch screen 117 so that the ultrasound image appears to smoothly rotate in a counter-clockwise direction as the fingers are rotated on the touch screen 117. According to the embodiment represented in FIG. 6, the multi-touch gesture of rotating two fingers results in rotating the ultrasound image displayed on the main screen 188; for example, the final image ultrasound image 182 is clearly rotated in a counter-clockwise direction from the initial ultrasound image 180. Performing a rotation with two fingers, or more than two fingers according to other embodiments, results in directly adjusting the ultrasound image by rotating the ultrasound image displayed on the main screen 118.

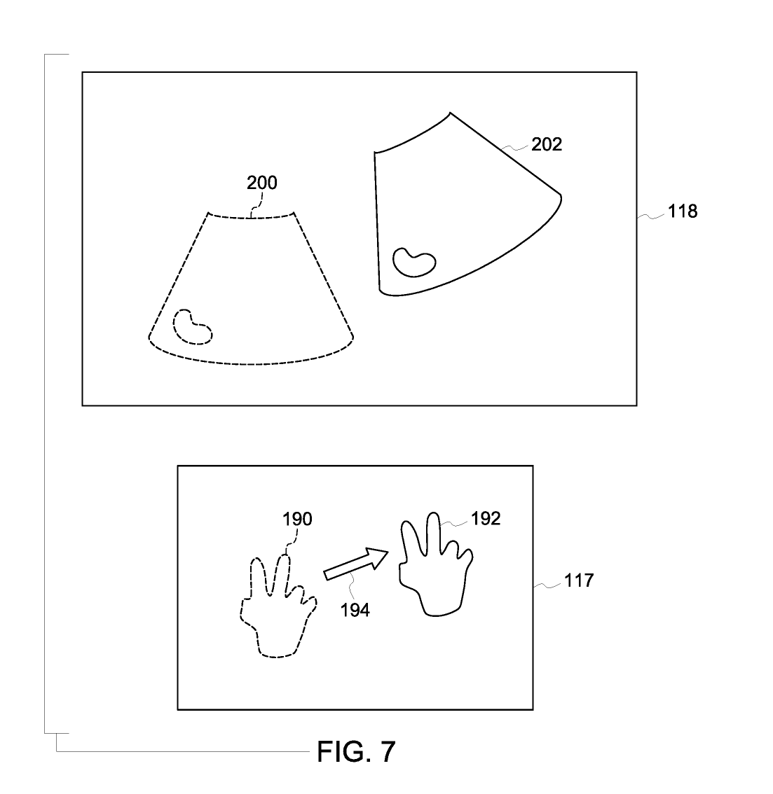

FIG. 7 is schematic representation of a touch screen and a main screen according to an embodiment where a multi-touch input comprises translating two fingers. FIG. 7 includes the touch screen 117 and the main screen 118. On the touch screen 117 the multi-touch gesture comprises translating the two fingers. A starting position 190 of the hand/fingers is shown in dashed line and a final position 192 is shown in solid line. An arrow 194 is used to show the movement of the fingers when performing this particular multi-touch gesture. Translating two fingers, or more than 2 fingers according to other embodiments, results in directly adjusting the ultrasound image by translating the ultrasound image in a corresponding direction. For example an initial ultrasound image 200 is shown in dashed line and a final ultrasound image 202 is shown in solid line. The initial ultrasound image 200 represents the ultrasound image that would be displayed before the multi-touch gesture is input through the touch screen 117. The final ultrasound image 202 represents the ultrasound image that is displayed after completing the multi-touch gesture, which in this exemplary embodiment comprises translating two fingers. It should be appreciated that the ultrasound image displayed on the main display 118 may be adjusted from the initial ultrasound image 200 to the final ultrasound 202 in real-time as the multi-touch gesture of translating the two fingers is input through the touch screen 117. It should be appreciated by those skilled in the art that the images displayed on the main screen 118 may smoothly transition from the initial image 200 to the final image 202. In other words, intermediate images may be displayed as the multi-touch gesture is being performed on the touch screen 117 so that the ultrasound image appears to smoothly translate across the main screen as the multi-touch gesture is translated across the touch screen 117. According to the embodiment represented in FIG. 7, the multi-touch gesture of translating the two fingers results in translating the ultrasound image displayed on the main screen 118; for example, the final ultrasound image 202 is clearly translated to the right with respect to the initial ultrasound image 200. Performing a translation with two fingers, or more than two fingers according to other embodiments, results in directly adjusting the ultrasound image by translating the ultrasound image displayed on the main screen 118.

Displaying a replication of the ultrasound image on the touch screen 117, such as would be performed at step 310, allows the user to have a better idea of how the specific multi-touch gesture will interact with the ultrasound image. According to an embodiment, both the ultrasound image displayed on the touch screen 117 and the ultrasound image displayed on the main screen 118 may both be adjusted and updated in real-time as the user inputs the multi-touch gesture. In other words, the multi-touch gesture may be used to adjust both the ultrasound image displayed on the main screen 118 and the ultrasound image displayed on the touch screen 117 at the same time. Displaying the ultrasound image on the touch screen may allow the user to manipulate the ultrasound image on the main screen more intuitively because the user can more easily see how their multi-touch gesture relates to the ultrasound image.

FIGS. 5, 6 and 7 show three exemplary multi-touch gestures, but it should be appreciated that any multi-touch gesture may be used to transition from the standard mode to the image-manipulation mode. The ultrasound images may be images of a volume rendering based on ultrasound data according to some embodiments. When manipulating/adjusting volume renderings, it may additionally be possible to control the rotation of the volume rendering about any axis in three-dimensional space with various multi-touch gestures.

According to an embodiment, the ultrasound imaging system 100 may stay in the image-manipulation mode for a predetermined amount of time after the multi-touch gesture has been completed. For example, the ultrasound imaging system 100 may stay in the image-manipulation mode for a few seconds, such as 2, 3, 4, or 5 seconds after the completion of the multi-touch gesture that initiated the transition from the standard mode to the image-manipulation mode. According to other embodiments, the predetermined time may be shorter than 2 seconds or longer than 5 seconds. The predetermined amount of time that the ultrasound imaging system 100 remains in the image-manipulation mode may also be an amount of time that is a non-integral number of seconds, and/or it may be user-selectable according to other embodiments.

In addition to switching back from the image-manipulation mode to the standard mode in response to a timeout, as described hereinabove, the ultrasound imaging system 100 may also switch back to the standard mode (i.e., de-activate the image-manipulation mode) in response to other inputs. For example, in other embodiments, the the ultrasound imaging system 100 may include a hardware key causing the switch from image-manipulation mode to standard mode. Embodiments may also include a soft key on the touch screen 117 causing the switch from image-manipulation mode to standard mode.

At step 314, the processor 116 determines if the predetermined amount of time has passed since the completion of the multi-touch gesture. If the predetermined amount of time has passed, such as 5 seconds according to an exemplary embodiment, the method 300 returns to step 302, where the ultrasound imaging system 100 reverts back to the standard mode.

If at step 314, the predetermined amount of time has not yet passed, the method 300 advances to step 316. At step 316, the touch screen 117 receives another gesture. The gesture may be a multi-touch gesture or, according to some embodiments, the gesture may also be a single-tough gesture. The gesture input through the touch screen 117 at step 316 directly adjusts the ultrasound image. According to some embodiments it may be possible to directly adjust the ultrasound image displayed on the mains screen 118 with either a single-touch or a multi-touch gesture while the system is in the image-manipulation mode.

If the predetermined amount of time passes from the last gesture input through the touchscreen 117, the system reverts back to the standard mode. For example, at 320, if the predetermined amount of time has passed since the last gesture was completed, then the method 300 returns to step 302. If the predetermined has not passed and another gesture is inputted through the touch screen 117, then the method returns to step 316 where the additional gesture is received and used to directly adjust the ultrasound image at step 318. Steps 316, 318, and 320 may be iterated as long as a new gesture is inputted before the predetermined amount of time has passed from the most-recently performed multi-touch gesture. If the predetermined amount of time has passed since the completion of the most-recently performed multi-touch gesture, then that system reverts back to standard mode.

Certain embodiments may provide a non-transitory computer readable medium having a stored thereon, a computer program having at least one code section that is executable by a machine for causing the machine to perform steps of the method 300 disclosed herein.

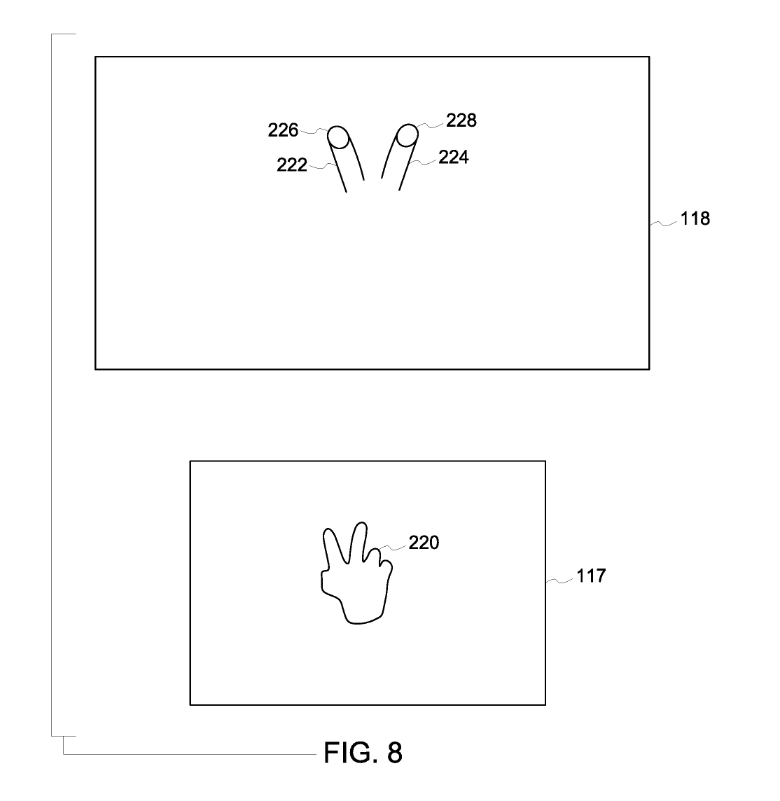

According to various embodiments, one or more icons may be displayed on the main screen 118 while performing the multi-touch gesture on touch screen 117. For example, FIG. 8 is a schematic representation of the touch screen 117 and the main screen 118 according to an embodiment where two icons are positioned on the main screen 118 to correspond to the multi-touch gesture performed on the touch screen 117. According to an embodiment, the touch screen 117 corresponds with the main screen 118, and the positions of the icons on the main screen 118 correspond to the positions of the user's fingers contacting the touch screen 117. This means that even if the touch screen 117 and the main screen 118 are different sizes, the icons are displayed on the same positions relative to the main screen as the user's fingers contacting the touch screen 117 are relative to the touch screen 117. FIG. 8 provides is an example of an exemplary embodiment. A user's hand 220 is schematically represented with respect to the touch screen 117. The user's hand 220 represents the position of the user's hand while the gesture is being input through the touch screen 117. A first icon 222 and a second icon 224 are displayed on the main screen 118 while the gesture is being input. According to the embodiment shown in FIG. 8, the first icon 222 is a representation of a first finger and the second icon 224 is a representation of a second finger. The embodiment shown in FIG. 8 also includes a representation of a first fingertip 226 and a representation of a second fingertip 228. In other embodiments, the icons may include only the representation of the fingertips. Additionally, icons of other shapes may be used to represent the positions of the fingertips according to other embodiments. The icons may comprise indicators according to various embodiments. For example, indicators may include an arrow, a circle, a square, or other shapes according to various embodiments. According to other embodiments, the icons may comprise a representation of at least two fingertips.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of controlling an ultrasound imaging system comprising both a main screen and a separate touch screen, the method comprising:
displaying an ultrasound image on the main screen;
displaying graphical user interface icons on the touch screen at the same time as said displaying the ultrasound image on the main screen while the ultrasound imaging system is in a standard mode, where, in the standard mode, the touch screen is used to interface with the graphical user interface icons displayed on the touch screen;
receiving a single-touch gesture through the touch screen while in the standard mode;
performing a command associated with one of the graphical user interface icons in response to the single-touch gesture while in the standard mode;
receiving a multi-touch gesture through the touch screen;
switching from operating in the standard mode to operating in an image-manipulation mode in response to the multi-touch gesture, where, in the image-manipulation mode, the touch screen is used to directly adjust the ultrasound image displayed on the main screen; and
directly adjusting the ultrasound image based on the multi-touch gesture while in the image-manipulation mode.

2. The method of claim 1, where the multi-touch gesture comprises a swipe with 2 or more fingers and directly adjusting the ultrasound image comprises translating the image.

3. The method of claim 1, where the multi-touch gesture comprises a rotation of two or more fingers and directly adjusting the ultrasound image comprises rotating the ultrasound image.

4. The method of claim 1, where the multi-touch gesture comprises pinching two or more fingers and directly adjusting the ultrasound image comprises zooming-out the ultrasound image.

5. The method of claim 1, where the multi-touch gesture comprises spreading two or more fingers and directly adjusting the ultrasound image comprises zooming-in the ultrasound image.

6. The method of claim 1, further comprising displaying the ultrasound image on the touch screen while in the image-manipulation mode.

7. The method of claim 1, further comprising changing back from the image-manipulation mode to the standard mode after a predetermined amount of time from a most-recently-completed multi-touch gesture.

8. The method of claim 1, further comprising displaying one or more icons on the main screen while performing the multi-touch gesture, where the touch screen corresponds with the main screen and the one or more icons are positioned on the main screen to correspond to the multi-touch gesture performed on the touch screen.

9. The method of claim 8, where the one or more icons comprise representations of at least two fingers.

10. The method of claim 8, where the one or more icons comprise representations of at least two fingertips.

11. The method of claim 8, where the one or more icons comprise at least two indicators.

12. The method of claim 8, further comprising moving the one or more icons on the main screen as the multi-touch gesture is performed.

13. An ultrasound imaging system comprising:
a probe configured to acquire ultrasound data;
a main screen;
a touch screen; and
a processor connected to the probe, the main screen and the touch screen, wherein the processor is configured to:
display an ultrasound image on the main screen, where the ultrasound image is based on the ultrasound data;
display graphical user interface icons on the touch screen at the same time as the ultrasound image is displayed on the main screen while operating in a standard mode, where, in the standard mode, the touch screen is used to interface with the graphical user icons on the touch screen;
perform a command associated with one of the graphical user interface icons in response to receiving a single-touch gesture through the touch screen while operating in the standard mode;
switch from operating in the standard mode to operating in an image-manipulation mode in response to receiving a multi-touch gesture through the touch screen, where, in the image-manipulation mode, the touch screen is used to directly adjust the ultrasound image displayed on the main screen; and
directly adjust the ultrasound image displayed on the main screen in response to the multi-touch gesture.

14. The ultrasound imaging system of claim 13, where the processor is configured to translate the ultrasound image on the main screen when the multi-touch gesture is a swipe of at least two fingers.

15. The ultrasound imaging system of claim 13, where the processor is configured to rotate the ultrasound image on the main screen when the multi-touch gesture is a rotation of at least two fingers.

16. The ultrasound imaging system of claim 13, where the processor is configured to zoom-in on the ultrasound image on the main screen when the multi-touch gesture comprises spreading a first finger apart from a second finger.

17. A non-transitory computer readable medium having stored thereon, a computer program having at least one code section, the at least one code section being executable by a machine for causing the machine to perform steps comprising:
displaying an ultrasound image on a main screen of an ultrasound imaging device while displaying graphical user interface icons on a touch screen of the ultrasound imaging device while in a standard mode, where, in the standard mode, the touch screen is used to interface with the graphical user interface icons;
performing a command associated with one of the graphical user interface icons in response to receiving a single-touch gesture selecting one of the graphical user interface icons while in the standard mode;
switching from operating in the standard mode to operating in an image-manipulation mode in response to receiving a multi-touch gesture through the touch screen, where, in the image-manipulation mode, the touch screen is used to directly adjust the ultrasound image displayed on the main screen; and
directly adjusting the ultrasound image based on the multi-touch gesture.

18. The non-transitory computer readable medium according to claim 17, comprising the step of switching from the image-manipulation mode to the standard mode after a predetermined amount of time from the completion of a most-recently-performed multi-touch gesture.

19. The non-transitory computer readable medium according to claim 17, where directly adjusting the ultrasound image comprises one of translating, rotating, zooming-in, and zooming-out in response to the multi-touch gesture.

20. The non-transitory computer readable medium according to claim 17, comprising the step of displaying the ultrasound image on the touch screen while in the image-manipulation mode.
